(19) United States
(12) Patent Application Publication
(10) Pub. No.: US 2003/0108577 A1
(43) Pub. Date: Jun. 12, 2003

(54) Cosmetic and/or dermatological acid composition containing an amphiphilic polymer

(76) Inventors: Raluca Lorant, Thiais (FR); Paula Lennon, Lyon (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/181,412

(22) PCT Filed: Jan. 8, 2002
(86) PCT No.: PCT/FR02/00047

(30) Foreign Application Priority Data
Jan. 11, 2001 (FR) 01/00336

(51) Int. Cl. A61K 7/00

(52) U.S. Cl. 424/401

(57) ABSTRACT

The present invention relates to a cosmetic and/or dermatological composition containing an acidic aqueous medium and at least one amphiphilic polymer including at least one ethylenically unsaturated monomer containing a sulphonic group, in free form or partially or totally neutralized form and comprising at least one hydrophobic portion.

The invention also relates to the use of this composition for cosmetically treating and/or making up keratin materials, especially the skin, the hair and the mucous membranes of the skin.

The invention also relates to the use of an amphiphilic polymer including at least one ethylenically unsaturated monomer containing a sulphonic group, in free form or partially or totally neutralized form and comprising at least one hydrophobic portion to stabilize a cosmetic or dermatological composition containing at least one acidic active agent and/or having a pH less than or equal to 5.

The present invention relates to a cosmetic and/or dermatological composition containing an acidic aqueous medium and at least one amphiphilic polymer including at least one ethylenically unsaturated monomer containing a sulphonic group, in free form or partially or totally neutralized form and comprising at least one hydrophobic portion.

The invention also relates to the use of this composition in particular for cosmetically treating and/or making up keratin materials, in particular the skin, the hair and the mucous membranes, and for treating acne.

The use of acidic agents such as hydroxy acids as active agents in cosmetic or dermatological compositions is known. These active agents are used in particular for caring for the face and/or the body and more specifically for giving the face a light, bright complexion and hence a good appearance, a smooth and younger look, for treating fine lines and wrinkles in the skin, for depigmenting the skin and, in particular, for removing the blemishes which appear with ageing, and for removing the comedones due to acne and for treating damaged hair, providing it with tone, giving it back strength and reinforcing the keratin fibres.

The compositions conventionally used in the cosmetic and/or dermatological fields are water-in-oil (W/O) emulsions, oil-in-water (O/W) emulsions or aqueous gels in which it is often difficult if not impossible to incorporate organic acidic active agents because the latter tend to destabilize the compositions in which they are present.

In effect, these acidic active agents have a general tendency to recrystallize or to degrade. This results in a greater or lesser loss of efficacy from these compositions, depending on the degree of recrystallization and/or degradation, which goes against the desired objective. Moreover, this recrystallization or degradation may modify the overall stability of these compositions and also their appearance and consistency (loss of viscosity and fluidification), which may cause the user to turn away from these compositions for specific treatment. In order to stabilize the emulsions comprising these acidic active agents, the addition of surfactants is known. However, the surfactants can cause problems of harmlessness and may aggravate any smarting and discomfort associated with the use of hydroxy acids and with the low pH.

It has already been envisaged in application EP-A-642 781 to use for this purpose acidic compositions in the form of oil-in-water-type emulsions comprising a substantially water-soluble, crosslinked anionic copolymer consisting of units deriving from the reaction between (i) acrylamide, (ii) 2-acrylamido-2-methylpropanesulphonic acid and (iii) at least one compound containing olefinic polyunsaturation. As a copolymer of this type mention may be made of Sepigel 305 sold by Seppic. These copolymers make it possible to stabilize the compositions comprising the acidic active agents. However, in order to obtain a good stabilizing effect, it is necessary to use fairly large proportions of gelling agent (greater than 2% or even than 4%) and, at these levels, the harmlessness and the comfort of these gelling agents are not always satisfactory.

Moreover, the polymers commonly used to stabilize the emulsions, such as carboxyvinyl polymers such as the Carbopol products, are incompatible with acids and lead to highly fluid and unstable gels and emulsions, owing to the fact that these polymers lose their thickening properties in an acidic medium.

Moreover, document EP-A-815845 describes the use of homopolymers of 2-acrylamido-2-methylpropane-sulphonic acid which are crosslinked and neutralized to the extent of at least 90% (AMPS polymers) and which make it possible to stabilize acidic media while retaining excellent cosmetic properties. However, the addition of a certain amount of acid causes the fluidification of the gels of these AMPS homopolymers, which may be detrimental if the aim is to obtain a fairly thick gel, and which may possibly cause a loss of the physicochemical stability of the composition, especially when it is in the form of an emulsion.

There is therefore a need for a stable composition in the form of an aqueous gel or an emulsion which can be used in particular in the cosmetic and/or dermatological fields and which allows sufficient incorporation of the acidic active agents which are generally used in these fields with the aim of maximum efficacy while retaining satisfactory viscosity and good stability.

The applicant has surprisingly discovered a new family of thickening and/or gelling polymers which make it possible to obtain cosmetic and dermatological compositions of acidic pH, which are able to obtain viscosities which are high and stable over time at ambient temperature or at higher temperatures. Furthermore, when the composition is in the form of an emulsion, it is stable even in the absence of emulsifiers which are conventionally used for stabilizing emulsions.

These polymers make it possible to produce stable acidic gels and emulsions which can be applied and which spread easily and homogeneously without leaving any fatty sensation, and which exhibit good cosmetic properties.

Thus, one subject of the present invention is a cosmetic and/or dermatological composition containing a physiologically acceptable acidic aqueous medium and at least one amphiphilic polymer including at least one ethylenically unsaturated monomer containing a sulphonic group, in free form or partially or totally neutralized form and comprising at least one hydrophobic portion.

In the present patent application, the expression “physiologically acceptable medium” means a medium that is compatible with all keratin materials, such as the skin, including the scalp, the nails, the mucous membranes, the eyes and the hair, or any other area of body skin. Moreover, this is an aqueous medium, that is to say a medium containing an amount of water of at least 5% by weight, preferably ranging from 30 to 99.4% by weight, and better still from 30 to 95% by weight, relative to the total weight of the composition.

In the present invention the term “acidic medium” refers to an aqueous medium with a pH of less than 6, in particular less than or equal to 5, and in practice selected within the range from 1 to 5. Beyond the value of pH 6, the formulation of acidic active agents no longer presents a difficulty since the acid is in an at least partially neutralized state, and in general indeed is totally neutralized.

A “cosmetic composition” means a composition which has a pleasant feel, appearance and taste.

A subject of the present invention is also the use of an amphiphilic polymer including at least one ethylenically unsaturated monomer containing a sulphonic group, in free form or partially or totally neutralized form and comprising at least one hydrophobic portion, to obtain a stable cosmetic or dermatological composition containing at least one acidic active agent and/or having a pH less than or equal to 5.

The expression “stable composition” means a composition whose viscosity remains virtually constant after addition of acid, that is to say a composition in which the addition of acid leads to a reduction in the viscosity to below 5%, and preferably to practically zero.

Other characteristics, features and advantages of the present invention will become apparent on reading the detailed description which will follow.

Amphiphilic Polymers According to the Invention

The polymers in accordance with the invention are amphiphilic polymers comprising at least one ethylenically unsaturated monomer containing a sulphonic group, in free form or partially or totally neutralized form and comprising at least one hydrophobic portion.

The expression “amphiphilic polymer” means any polymer comprising both a hydrophilic portion and a hydrophobic portion and especially a fatty chain.

The hydrophobic portion present in the polymers of the invention preferably contains from 6 to 50 carbon atoms, more preferably from 6 to 22 carbon atoms, even more preferably from 6 to 18 carbon atoms and more particularly from 12 to 18 carbon atoms.

Preferably, the polymers in accordance with the invention are partially or totally neutralized with a mineral base (sodium hydroxide, potassium hydroxide or aqueous ammonia) or an organic base such as mono-, di- or triethanolamine, an aminomethylpropanediol, N-methylglucamine, basic amino acids, for instance arginine and lysine, and mixtures of these compounds.

The amphiphilic polymers in accordance with the invention generally have a number-average molecular weight ranging from 1000 to 20 000 000 g/mol, preferably ranging from 20 000 to 5 000 000 and even more preferably from 100 000 to 1 500 000 g/mol.

The amphiphilic polymers according to the invention may or may not be crosslinked. Crosslinked amphiphilic polymers are preferably chosen.

When they are crosslinked, the crosslinking agents may be chosen from compounds containing olefinic polyunsaturation commonly used for the crosslinking of polymers obtained by free-radical polymerization.

Mention may be made, for example, of divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylol propane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allyl or vinyl ethers of polyfunctional alcohols, and also allylic esters of phosphoric and/or vinylphosphonic acid derivatives, or mixtures of these compounds.

Methylenebisacrylamide, allyl methacrylate or trimethylolpropane triacrylate (TMPTA) will be used more particularly. The degree of crosslinking will generally range from 0.01 mol % to 10 mol % and more particularly from 0.2 mol % to 2 mol % relative to the polymer.

The ethylenically unsaturated monomers containing a sulphonic group are chosen especially from vinylsulphonic acid, styrenesulphonic acid, (meth)acrylamido(C₁-C₂₂)alkylsulphonic acids, N-(C₁-C₂₂)alkyl(meth)acrylamido(C₁-C₂₂)alkylsulphonic acids, for instance undecylacrylamidomethanesulphonic acid, and also partially or totally neutralized forms thereof. (Meth) acrylamido (C₁-C₂₂) alkylsulphonic acids such as, for example, acrylamidomethanesulphonic acid, acrylamidoethanesulphonic acid, acrylamidopropane-sulphonic acid, 2-acrylamido-2-methylpropanesulphonic acid, methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid or 2-acrylamido-2,6-dimethyl-3-heptanesulphonic acid, and also partially or totally neutralized forms thereof, will more preferably be used.

2-Acrylamido-2-methylpropanesulphonic acid (AMPS), and also its partially or totally neutralized forms, will more particularly be used.

The amphiphilic polymers in accordance with the invention may be chosen especially from random amphiphilic polymers of AMPS modified by reaction with a C₆-C₂₂ n-monoalkylamine or di-n-alkylamine, and such as those described in patent application WO-A-00/31154 (forming an integral part of the content of the description). These polymers may also contain other ethylenically unsaturated hydrophilic monomers chosen, for example, from (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

The preferred polymers of the invention are chosen from amphiphilic copolymers of AMPS and of at least one ethylenically unsaturated hydrophobic monomer comprising at least one hydrophobic portion containing from 6 to 50 carbon atoms, more preferably from 6 to 22 carbon atoms, even more preferably from 6 to 18 carbon atoms and more particularly 12 to 18 carbon atoms.

These same copolymers may also contain one or more ethylenically unsaturated monomers not comprising a fatty chain, such as (meth)acrylic acids, β-substituted alkyl derivatives thereof or esters thereof obtained with monoalcohols or mono- or polyalkylene glycols, (meth)acrylamides, vinylpyrrolidone, maleic anhydride, itaconic acid or maleic acid, or mixtures of these compounds.

These copolymers are described especially in patent application EP-A-750 899, patent US-A-5 089 578 and in the following publications from Yotaro Morishima:

“Self-assembling amphiphilic polyelectrolytes and their nanostructures—Chinese Journal of Polymer Science Vol. 18, No. 40, (2000), 323-336”;

“Micelle formation of random copolymers of sodium 2-(acrylamido)-2-methylpropanesulfonate and a non-ionic surfactant macromonomer in water as studied by fluorescence and dynamic light scattering—Macromolecules 2000, Vol. 33, No. 10—3694-3704”;

“Solution properties of micelle networks formed by non-ionic moieties covalently bound to a polyelectrolyte: salt effects on rheological behavior—Langmuir, 2000, Vol. 16, No. 12, 5324-5332”;

“Stimuli responsive amphiphilic copolymers of sodium 2-(acrylamido)-2-methylpropanesulfonate and associative macromonomers—Polym. Preprint, Div. Polym. Chem. 1999, 40(2), 220-221”.

The ethylenically unsaturated hydrophobic monomers of these particular copolymers are preferably chosen from the acrylates or acrylamides of formula (I) below:

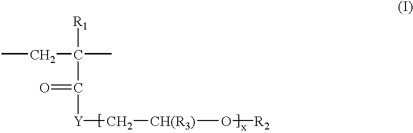

in which R₁ and R₃, which may be identical or different, denote a hydrogen atom or a linear or branched C₁-C₆ alkyl radical (preferably methyl); Y denotes O or NH; R₂ denotes a hydrophobic hydrocarbon-based radical containing at least from 6 to 50 carbon atoms and more preferably from 6 to 22 carbon atoms and even more preferably from 6 to 18 carbon atoms and more particularly from 12 to 18 carbon atoms; x denotes a number of moles of alkylene oxide and ranges from 0 to 100.

The radical R₂ is preferably chosen from linear C₆-C₁₈ alkyl radicals (for example n-hexyl, n-octyl, n-decyl, n-hexadecyl and n-dodecyl) and branched or cyclic C₆-C₁₈ alkyl radicals (for example cyclododecane (C₁₂) or adamantane (C₁₀)) ; C₆-C₁₈ alkylperfluoro radicals (for example the group of formula —(CH₂)₂—(CF₂)₉—CF₃); the cholesteryl radical (C₂₇) or a cholesterol ester residue, for instance the cholesteryl oxyhexanoate group; aromatic polycyclic groups, for instance naphthalene or pyrene. Among these radicals, the ones that are more particularly preferred are linear alkyl radicals and more particularly the n-dodecyl or n-hexadecyl radical.

According to one particularly preferred form of the invention, the monomer of formula (I) comprises at least one alkylene oxide unit (x≧1) and preferably a polyoxyalkylenated chain. The polyoxyalkylenated chain preferably consists of ethylene oxide units and/or of propylene oxide units and even more particularly consists of ethylene oxide units. The number of oxyalkylenated units (or moles of alkylene oxide) generally ranges from 3 to 100, more preferably from 3 to 50 and even more preferably from 7 to 25.

Among these polymers, mention may be made of:

crosslinked or non-crosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer, such as those described in patent application EP-A-750 899;

terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units, such as those described in patent US-5 089 578.

Mention may also be made of copolymers of totally neutralized AMPS and of dodecyl or n-hexadecyl methacrylate, and also crosslinked and non-crosslinked copolymers of AMPS and of n-dodecylmethacrylamide, such as those described in the Morishima articles mentioned above.

Mention will be made more particularly of the copolymers consisting of 2-acrylamido-2-methylpropanesulphonic acid (AMPS) units of formula (II) below:

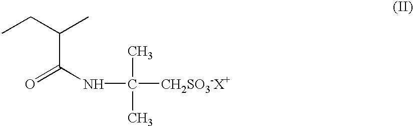

in which X⁺ is a proton, an alkali metal cation, an alkaline-earth metal cation or the ammonium ion, and of units of formula (III) below:

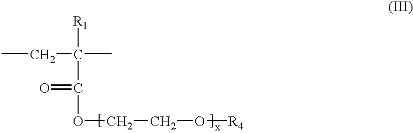

in which x denotes an integer ranging from 3 to 100, preferably from 5 to 80 and more preferably from 7 to 25; R₁ has the same meaning as that given above in formula (I) and R₄ denotes a linear or branched C₆-C₂₂ and more preferably C₁₀-C₂₂ alkyl.

The polymers that are particularly preferred are those for which in formula (III) x=25, R₁ denotes methyl and R₄ represents n-dodecyl; they are described in the Morishima articles mentioned above. Other preferred polymers are those for which x=25, R₁ denotes methyl and R₄ represents n-hexadecyl (C₁₆) or n-octadecyl (C₁₈) or mixtures thereof.

The polymers for which X⁺ denotes sodium or ammonium are more particularly preferred.

The preferred amphiphilic polymers in accordance with the invention may be obtained according to the standard free-radical polymerization processes in the presence of one or more initiators such as, for example, azobisisobutyronitrile (AIBN), azobisdimethylvaleronitrile, 2,2-azobis[2-amidinopropane] hydrochloride (ABAH=2,2-AzoBis[2-Amidinopropane] Hydrochloride), organic peroxides such as dilauryl peroxide, benzoyl peroxide, tert-butyl hydroperoxide, etc., mineral peroxide compounds such as potassium persulphate or ammonium persulphate, or H₂O₂ optionally in the presence of reducing agents.

The amphiphilic polymers are obtained especially by free-radical polymerization in tert-butanol medium in which they precipitate. Using precipitation polymerization in tert-butanol, it is possible to obtain a size distribution of the polymer particles that is particularly favourable for its uses.

The size distribution of the polymer particles may be determined, for example, by laser diffraction or image analysis. An advantageous distribution for this type of polymer, determined by image analysis, is as follows: 60.2% less than 423 microns, 52.0% less than 212 microns, 26.6% less than 106 microns, 2.6% less than 45 microns and 26.6% greater than 850 microns.

The reaction may be performed at a temperature of between 0 and 150° C., preferably between 10 and 100° C., either at atmospheric pressure or under reduced pressure. It may also be performed under inert atmosphere, and preferably under nitrogen.

According to this process 2-acrylamido-2-methylpropanesulphonic acid (AMPS) or a sodium or ammonium salt thereof was especially polymerized with a (meth)acrylic acid ester and

a C₁₀-C₁₈ alcohol oxyethylenated with 8 mol of ethylene oxide (Genapol® C-080 from the company Hoechst/Clariant),

a C₁₁ oxo alcohol oxyethylenated with 8 mol of ethylene oxide (Genapol® UD-080 from the company Hoechst/Clariant),

a C₁₁ oxo alcohol oxyethylenated with 7 mol of ethylene oxide (Genapol® UD-070 from the company Hoechst/Clariant),

a C₁₂-C₁₄ alcohol oxyethylenated with 7 mol of ethylene oxide (Genapol® LA-070 from the company Hoechst/Clariant),

a C₁₂-C₁₄ alcohol oxyethylenated with 9 mol of ethylene oxide (Genapol® LA-090 from the company Hoechst/Clariant),

a C₁₂-C₁₄ alcohol oxyethylenated with 11 mol of ethylene oxide (Genapol® LA-110 from the company Hoechst/Clariant),

a C₁₆-C₁₈ alcohol oxyethylenated with 8 mol of ethylene oxide (Genapol® T-080 from the company Hoechst/Clariant),

a C₁₆-C₁₈ alcohol oxyethylenated with 15 mol of ethylene oxide (Genapol® T-150 from the company Hoechst/Clariant),

a C₁₆-C₁₈ alcohol oxyethylenated with 11 mol of ethylene oxide (Genapol® T-110 from the company Hoechst/Clariant),

a C₁₆-C₁₈ alcohol oxyethylenated with 20 mol of ethylene oxide (Genapol® T-200 from the company Hoechst/Clariant),

a C₁₆-C₁₈ alcohol oxyethylenated with 25 mol of ethylene oxide (Genapol® T-250 from the company Hoechst/Clariant),

a C₁₈-C₂₂ alcohol oxyethylenated with 25 mol of ethylene oxide and/or a C₁₆-C₁₈ iso alcohol oxyethylenated with 25 mol of ethylene oxide.

The molar % concentration of the units of formula (II) and of the units of formula (III) in the polymers according to the invention will vary as a function of the desired cosmetic use and of the desired rheological properties of the formulation. It may range between 0.1 mol % and 99.9 mol %.

Preferably, for the most hydrophobic polymers, the molar proportion of units of formula (I) or (III) ranges from 50.1% to 99.9%, more particularly from 70% to 95% and even more particularly from 80% to 90%.

Preferably, for the sparingly hydrophobic polymers, the molar proportion of units of formula (I) or (III) ranges from 0.1% to 50%, more particularly from 5% to 25% and even more particularly from 10% to 20%.

The monomer distribution in the polymers of the invention may be, for example, alternating, block (including multiblock) or random.

According to the invention, it is preferable for the polymers to contain heat-sensitive pendant chains and for the aqueous solution thereof to have a viscosity that, beyond a certain threshold temperature, increases or remains virtually constant as the temperature increases.

Even more particularly, the preferred polymers are those whose aqueous solution has a viscosity that is low below a first threshold temperature and that, above this first threshold temperature, increases to a maximum as the temperature increases, and that, above a second threshold temperature, decreases again as the temperature increases. From this perspective, it is preferable for the viscosity of the polymer solutions below the first threshold temperature to be from 5% to 50%, in particular from 10% to 30% of the maximum viscosity at the second threshold temperature.

These polymers preferably lead in water to a phenomenon of demixing by heating, reflected by curves showing, as a function of the temperature and the concentration, a minimum known as the LCST (Lower Critical Solution Temperature).

The viscosities (measured at 25° C. using a Brookfield viscometer, needle No. 7) of the aqueous 1% solutions preferably range from 20 000 mPa.s to 100 000 mpa.s and more particularly from 60 000 mPa.s to 70 000 mPa.s.

The amphiphilic polymers in accordance with the invention are present in the compositions in concentrations ranging from 0.01% to 30% by weight of active material, more preferably from 0.1% to 10%, even more preferably from 0.1% to 5% by weight and even more particularly from 0.2% to 2% by weight of active material relative to the total weight of the composition.

The compositions according to the invention may comprise cosmetic and/or dermatological acidic active agents which are stabilized in the acidic aqueous medium in the presence of one or more amphiphilic polymers as defined above.

As acidic active agents which can be used in the composition of the invention mention may be made of hydroxy acids, which may be α-hydroxy acids or β-hydroxy acids, and which may be linear, branched or cyclic, saturated or unsaturated. The hydrogen atoms of the carbon chain may further be substituted by halogen, alkyl, acyl, acyloxy, alkoxycarbonyl or alkoxy radicals having from 2 to 18 carbon atoms; α-keto acids; β-keto acids; their derivatives, and mixtures thereof.

As acidic active agents mention may be made more particularly of ascorbic acid, kojic acid; caffeic acid; salicylic acid and its derivatives, especially alkylated derivatives (for example n-octanoyl-5-salicylic acid, n-decanoyl-5-salicylic acid or n-dodecanoyl-5-salicylic acid, 2-hydroxy-3-methylbenzoic acid, 2-hydroxy-3-methoxybenzoic acid); β-hydroxy acids such as citric acid, lactic acid, methyllactic acid, glucuronic acid, glycolic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid; mandelic acid; benzoic acid; phenyllactic acid; gluconic acid; galacturonic acid; aleuritic acid; ribonic acid; tartronic acid; tartaric acid; malic acid; fumaric acid; retinoic acid and its derivatives; benzene-1,4-di(3-methylidene-10-camphorsulphonic)acid; urocanic acid; 2-phenylbenzimidazole-5-sulphonic acid; α-(2-oxo-3-bornylidene)-4-toluenesulphonic acid; 2-hydroxy-4-methoxy-5-sulphonic acid; all of the natural or synthetic compounds containing such acids, such as plant extracts and, more specifically, fruit extracts; acidic xanthine derivatives (caffeine, theophylline); β-glycyrrhetinic acid; and asiatic acid. It is also possible to use mixtures of these various acidic active agents.

The acid or acids may be present in an amount ranging for example from 0.01 to 20% by weight, preferably from 0.1 to 10% and better still from 0.1 to 5% by weight relative to the total weight of the composition.

The physiologically acceptable medium of the compositions according to the invention is composed more particularly of water and, where appropriate, physiologically acceptable organic solvents. When the composition is in emulsion form, this medium further comprises an oily phase. The amount of water may range for example from 5 to 99.94% by weight, preferably from 30 to 99.94% and better still from 30 to 95% by weight relative to the total weight of the composition.

The organic solvents may represent from 0.2% to 50% of the total weight of the composition. They may be selected from the group consisting of hydrophilic organic solvents, lipophilic organic solvents, amphiphilic solvents or mixtures thereof.

Among the aqueous organic solvents mention may be made, for example, of linear or branched lower monoalcohols having from 1 to 8 carbon atoms such as ethanol, propanol, butanol, isopropanol and isobutanol; polyethylene glycols having from 6 to 80 ethylene oxide units; polyols such as propylene glycol, isoprene glycol, butylene glycol, glycerol and sorbitol; mono-or dialkyl isosorbides whose alkyl groups have from 1 to 5 carbon atoms such as dimethyl isosorbide; glycol ethers such as diethylene glycol monomethyl or monoethyl ether and propylene glycol ethers such as dipropylene glycol methyl ether; and sugars such as glucose.

As amphiphilic organic solvents mention may be made of polyols such as polypropylene glycol (PPG) derivatives, especially the esters of polypropylene glycol and fatty acid or of PPG and fatty alcohol, such as PPG-23 oleyl ether and PPG-36 oleate.

As lipophilic organic solvents mention may be made, for example, of fatty esters such as diisopropyl adipate, dioctyl adipate and alkyl benzoates.

In order to make the cosmetic or dermatological compositions of the invention more pleasant to use (softer on application, providing greater nutrition, more emollient), it is possible to add an oily phase (or fatty phase) to the medium of these compositions. This oily phase contains one or more oils, namely organic substances which are liquid at ambient temperature (20 to 25° C.) and at atmospheric pressure (760 mmHg).

When present the oily phase represents preferably from 0.1% to 50% of the total weight of the composition, better still from 1 to 30% and even better still from 5 to 20% of the total weight of the composition.

As oils which can be used in the composition of the invention, mention may be made for example of:

hydrocarbon-based oils of animal origin, such as perhydrosqualene;

hydrocarbon-based plant-origin oils such as liquid triglycerides of fatty acids containing from 4 to 10 carbon atoms, such as heptanoic or octanoic acid triglycerides or alternatively, for example, sunflower oil, corn oil, soya bean oil, marrow oil, grapeseed oil, sesame oil, hazelnut oil, apricot oil, macadamia oil, arara oil, castor oil, avocado oil, caprylic/capric acid triglycerides such as those sold by the company Stearineries Dubois or those sold under the names Miglyol 810, 812 and 818 by the company Dynamit Nobel, jojoba oil or karite butter;

synthetic esters and ethers in particular of fatty acids, such as the oils of formulae R¹COOR² and R¹OR² in which R¹ represents a fatty acid residue containing from 8 to 29 carbon atoms and R2 represents a branched or unbranched hydrocarbon-based chain containing from 3 to 30 carbon atoms, such as, for example, purcellin oil, isononyl isononanoate, isopropyl myristate, 2-ethylhexyl palmitate, 2-octyldodecyl stearate, 2-octyldodecyl erucate or isostearyl isostearate; hydroxylated esters such as isostearyl lactate, octyl hydroxystearate, octyldodecyl hydroxystearate, diisostearyl malate, triisocetyl citrate, and fatty alcohol heptanoates, octanoates and decanoates; polyol esters such as propylene glycol dioctanoate, neopentyl glycol diheptanoate and diethylene glycol diisononanoate; and pentaerythritol esters such as pentaerythrityl tetraisostearate;

linear or branched hydrocarbons of mineral or synthetic origin, such as volatile or non-volatile liquid paraffins and derivatives thereof, petroleum jelly, polydecenes or hydrogenated polyisobutene such as Parleam® oil;

fatty alcohols containing from 8 to 26 carbon atoms, such as cetyl alcohol, stearyl alcohol, and the mixture thereof (cetylstearyl alcohol), octyldodecanol, 2-butyloctanol, 2-hexyldecanol, 2-undecylpentadecanol oleyl alcohol, or linoleyl alcohol;

partially hydrocarbon-based and/or silicone-based fluoro oils such as those described in document JP-A-2 295 912;

silicone oils such as volatile or non-volatile polymethylsiloxanes (PDMSs) containing a linear or cyclic silicone-based chain, which are liquid or pasty at room temperature, in particular cyclopolydimethylsiloxanes (cyclomethicones) such as cyclohexasiloxane; polydimethylsiloxanes comprising alkyl, alkoxy or phenyl groups, pendant or at the end of a silicone-based chain, these groups containing from 2 to 24 carbon atoms; phenylsilicones such as phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and polymethylphenylsiloxanes;

mixtures thereof. “Hydrocarbon-based oil” is understood to mean, in the list of oils mentioned above, any oil containing a majority of carbon and hydrogen atoms and optionally ester, ether, fluoro, carboxylic acid and/or alcohol groups.

The other fatty substances which may be present in the oily phase are, for example, fatty acids containing from 8 to 30 carbon atoms, for instance stearic acid, lauric acid or palmitic acid; waxes, for instance lanolin, beeswax, carnauba wax, candelilla wax, paraffin wax, lignite wax or microcrystalline waxes, ceresine or ozokerite, synthetic waxes, for instance polyethylene waxes and Fischer-Tropsch waxes; gums such as silicone gums (dimethiconol); silicone resins such as trifluoromethyl-C1-4-alkyldimethicone and trifluoropropyldimethicone; and silicone elastomers, for instance the products sold under the names “KSG” by the company Shin-Etsu, under the names “Trefil”, “BY29” or “EPSX” by the company Dow Corning or under the names “Gransil” by the company Grant Industries.

These fatty substances may be chosen in a varied manner by a person skilled in the art in order to prepare a composition having the desired properties, for example of consistency or of texture.

Conventionally, the compositions of the invention may comprise adjuvants which are common in the cosmetic and dermatological fields, other conventional, aqueous or lipophilic gelling agents and/or thickeners; hydrophilic or lipophilic active agents; preservatives; antioxidants; perfumes; moisturizers; depigmenting agents; keratolytic agents; emulsifiers; vitamins; emollients; sequestrants; surfactants; polymers; basifying or acidifying agents; fillers; free-radical scavengers; ceramides; sunscreens (especially ultraviolet sunscreens); insect repellents; thinners; colorants (such as pigments, soluble dyes or nacres); bactericides; antidandruff agents. The amounts of these various adjuvants are those which are commonly used in the fields under consideration.

The person skilled in the art will of course take care to select the compound or compounds that are possibly to be added to the composition according to the invention in such a way that the advantageous properties intrinsically linked to the composition in accordance with the invention are not, or not substantially, adversely affected by the intended addition.

The compositions according to the invention may be present in any form appropriate for topical application, in particular in the form of solutions of the lotion or serum type, in the form of aqueous gels, in the form of emulsions obtained by dispersing a fatty phase in an aqueous phase (O/W) or inversely (W/O) or in the form of multiple emulsions (O/W/O or W/O/W), with a consistency which is liquid or semiliquid such as milks and more or less unctuous creams, cream gels, and pastes. They may optionally be packaged as an aerosol and may be present in the form of a mousse or spray. These compositions are prepared in accordance with the customary methods.

The composition of the invention may have a viscosity which is adapted to the product which it is desired to obtain. This viscosity may range for example from 5 to 50 poises (0.5 to 5 Pa·s), measured at ambient temperature (approximately 25° C.) using a Rheomat 180 and a 2, 3, 4 or 5 spindle according to the viscosity, at 200 s⁻¹.

The composition according to the invention may further comprise at least one oily phase (or fatty phase).

When the composition is an emulsion, the oily phase comprises one or more oils and optionally other fatty substances, as described above. The proportion of the oily phase in the emulsion may range for example from 5 to 80% by weight, and preferably from 5 to 50% by weight, relative to the total weight of the composition. The aqueous phase makes up the remainder to 100% by weight.

In one particular embodiment of the invention, advantageously, the emulsions prepared with the polymers according to the invention may be devoid of emulsifiers while being stable on storage irrespective of the temperature (for example between 5 and 50° C.).

The composition according to the invention may be used in many cosmetic or dermatological applications in which the presence of acidic active agents is useful; it may especially be used for treating, caring for and/or making up facial and/or body skin, mucous membranes (lips), the scalp and/or keratin fibres (hair or eyelashes), for example to give the face a light, bright complexion, good appearance, a smooth and younger look, for treating fine lines and wrinkles in the skin, for depigmenting the skin and, in particular, for removing the blemishes of ageing, for making comedones disappear, for treating damaged hair, providing it with tone, giving it back strength, and reinforcing the keratin fibres.

Thus, the compositions of the invention may be used as care products and/or hygiene products, such as protective, treatment or care creams for the face, for the hands or for the body, protective or care body milks, lotions, gels or mousses to care for the skin and mucous membranes or for cleansing, removing make-up from or scrubbing skin. They may also constitute make-up products for keratin fibres, the skin, the lips and/or the nails. In particular, they may constitute a foundation, a face powder, an eyeshadow, a lipstick, a mascara or an eyeliner.

The compositions of the invention may also be used as antisun products for protecting the skin against UV rays.

The compositions according to the invention may be used as hair products which are employed with or without rinsing, especially for washing, caring for, conditioning and maintaining the hairstyle or for shaping keratin fibres such as the hair. They may also comprise solid preparations which constitute soaps or cleansing bars.

The compositions of the invention may also be used as a product for buccodental care, such as as dentifrice pastes.

They may also be styling products such as setting lotions, brushing lotions, fixing compositions and styling compositions. The lotions may be packaged in various forms, in particular in vaporizers, pump flasks or aerosol containers, providing for application of the composition in vaporized form or in the form of a mousse. Packaging forms of this kind are indicated, for example, when the desire is to obtain a spray or a foam for fixing or treating the hair.

Thus, one subject of the present invention is also the cosmetic use of the cosmetic composition according to the invention, to treat, care for and/or make up the facial and/or body skin, mucous membranes (lips), the scalp and/or keratin fibres.

Another subject of the invention is a cosmetic process for treating human keratin materials such as the skin, including the scalp, the hair, the eyelashes, the eyebrows, the nails or the mucous membranes, especially the lips, characterized in that a cosmetic composition as defined above is applied to the keratin materials according to the usual technique for using this composition. For example: application of creams, gels, sera, lotions or milks to the skin, the scalp and/or the mucous membranes. The type of treatment depends on the acidic active agents present in the composition.

A further subject of the invention is the use of the above composition for preparing a pommade or ointment for treating the face and/or human body, including the hands, particularly for treating acne.

### Preparation Examples Preparation of ethoxylated (meth)acrylic esters:

These may especially be obtained by the action of glycidyl (meth)acrylate or (meth)acrylic acid or an alkyl (meth)acrylate or a (meth)acryloyl halide on an ethoxylated fatty alcohol. Non-limiting examples which may be mentioned include the following preparations:

a) starting with glycidyl methacrylate and Genapol T-250;

b) starting with (meth)acrylic acid and Genapol UD-070;

c) starting with methyl (meth)acrylate and Genapol LA-090;

d) starting with (meth)acryloyl chloride and Genapol UD-070.

a) 500 g of Genapol T-250 and 75 g of glycidyl methacrylate are placed in a one-litre three-necked reactor equipped with a stirrer, a thermometer and a reflux condenser. The reaction mixture is heated at a temperature of 100° C. for 2 hours and the excess glycidyl methacrylate is removed by distillation under reduced pressure. The monomer obtained may be used for the polymerization without further purification.

b) 500 g of Genapol UD-070, 100 g of (meth)acrylic acid and p-toluenesulphonic acid as catalyst are placed in a one-litre three-necked reactor equipped with a stirrer, a thermometer and a reflux condenser. The reaction mixture is refluxed for 2 hours and the excess acid and the water formed during the reaction are separated out by distillation under reduced pressure. The monomer obtained may be used for the polymerization without further purification.

c) 500 g of Genapol LA-090, 100 g of methyl (meth)acrylate and 20 g of titanium tetraisopropoxide are placed in a one-litre three-necked reactor equipped with a stirrer, a thermometer and a reflux condenser. The reaction mixture is refluxed for 2 hours and, after separation by distilling off the alcohol formed, the remaining ester is distilled under reduced pressure. The monomer obtained may be used for the polymerization without further purification.

d) 500 g of Genapol UD-070, 110 g of (meth)acryloyl chloride and 50 g of sodium carbonate are placed in a one-litre three-necked reactor equipped with a stirrer, a thermometer and a reflux condenser. The reaction mixture is refluxed for 2 hours and the excess acid chloride is separated out by distillation under reduced pressure. The monomer obtained may be used for the polymerization without further purification.

Polymerization According to the Precipitation Method in Tert-butanol

500 ml of tert-butanol and the calculated amount of AMPS are placed in a 2-litre reactor equipped with a reflux condenser, a gas inlet, a thermometer and a stirrer. The mixture is neutralized by introducing NH₃, and the monomer prepared above is added to the reaction mixture. The reaction mixture is made inert by passing nitrogen or argon through, and, when the internal temperature has reached 60° C., the initiator (AIBN) is introduced to initiate the polymerization. After a few minutes, the polymer thus prepared precipitates. The mixture is maintained at reflux for 2 hours, and the polymer is separated from the solvent by vacuum filtration and then dried under reduced pressure.

The following polymers were prepared in the manner described above: (starting with the following reagents in amounts expressed in grams)

| | | | | |
|---|---|---|---|---|
| Genapol T-250 methacrylate | 10 | 20 | 30 | 97 |
| AMPS neutralized with NH₃ | 90 | 80 | 90 | 3 |
| Methylenebisacrylamide | | | 1.5 | |
| (crosslinking agent) | | | | |
| Allyl methacrylate | | 1.7 | | |
| (crosslinking agent) | | | | |
| TMPTA (crosslinking agent) | 1.8 | | | 1.8 |
| Azobisisobutyronitrile | | | 1 | |
| (initiator) | | | | |
| Dilauryl peroxide (initiator) | 1 | 1 | | 1 |
| tert-Butanol | 300 | 300 | 300 | 300 |

The following were prepared in the same manner:

a copolymer crosslinked with allyl methacrylate, consisting of 80% by weight of AMPS units neutralized with NH₃, and of 20% by weight of Genapol T-250 methacrylate units [units of formula (III) in which R₁═CH₃, R₄═C₁₆-C₁₈ and x=25];

a copolymer crosslinked with allyl methacrylate, consisting of 90% by weight of AMPS units neutralized with NH₃, and of 10% by weight of Genapol LA-070 methacrylate units [units of formula (III) in which R₁═H, R₄═C₁₂-C₁₄ and x=7];

a TMPTA-crosslinked copolymer consisting of 90% by weight of AMPS units neutralized with NH₃, and of 10% by weight of Genapol T-200 methacrylate units [units of formula (III) in which R₁═CH₃, R₄═C₁₆-C₁₈ and x=20].

### Examples of compositions

The examples which follow illustrate the invention without being limiting in nature. The amounts are indicated as percentages by weight, except where otherwise mentioned.

### EXAMPLE 1

### Care Cream for a Bright Complexion

| | |
|---|---|
| Aqueous phase | |
| Crosslinked AMPS copolymer (*) | 2% |
| Mixture of fruit acids (lactic, tartaric | 1% |
| and malic) | |
| Triethanolamine | qs pH 3.5 |
| Preservative | qs |
| Water | qs 100% |
| Oily phase | |
| Mineral oil | 5% |
| Cyclohexasiloxane | 5% |
| Preservative | qs |

(*) Copolymer crosslinked with allyl methacrylate, consisting of 90% by weight of AMPS units neutralized with NH₃, and of 10% by weight of Genapol LA-070 methacrylate units [units of formula (III) in which R₁═H, R₄═C₁₂-C₁₄ and x = 7].

Procedure: the two phases are prepared separately and the oily phase is added to the aqueous phase with stirring.

This gives a creamy O/W emulsion which is very soft and comfortable on the skin.

### EXAMPLE 2

### Exfoliating Moisturizing Gel

| | |
|---|---|
| Crosslinked AMPS copolymer (*) | 2% |
| Mixture of fruit acids (lactic, tartaric | 1% |
| and malic) | |
| Preservative | qs |
| Water | qs 100% |

(*) Copolymer crosslinked with TMPTA, consisting of 90% by weight of AMPS units neutralized with NH₃, and of 10% by weight of Genapol T-200 methacrylate units [units of formula (III) in which R₁═CH₃, R₄═C₁₆-C₁₈ and x ° 20].

Procedure: hot and with stirring, the copolymer is added to the solution of the mixture of fruit acids and water. This gives a thick and creamy gel with a pH of 2.8. The stability of this gel is very good.

### EXAMPLE 3

### Foundation

| | |
|---|---|
| Crosslinked AMPS copolymer (*) | 2% |
| Oxides of iron and of titanium, coated with | 7% |
| PDMS | |
| Mixture of silicone gum and volatile | 16% |
| silicone (polydiphenyldimethylsiloxane + | |
| cyclopentadimethylsiloxane) | |
| Citric acid | 1% |
| Preservatives | 1% |
| Triethanolamine | qs pH 3.5 |
| Water | qs 100% |

(*) Copolymer crosslinked with allyl methacrylate, consisting of 80% by weight of AMPS units neutralized with NH₃, and of 20% by weight of Genapol T-250 methacrylate units [units of formula (III) in which R₁═CH₃, R₄═C₁₆-C₁₈ and x = 25]

A stable composition is obtained with a pH of 3.5 which allows a uniform and natural make-up to be obtained. The product is soft on application and easy to apply.

### EXAMPLE 4

### Foundation

| | |
|---|---|
| Crosslinked AMPS copolymer (*) | 2% |
| Oxides of iron and of titanium, coated with | 7% |
| PDMS | |
| Mixture of silicone gum and volatile | 16% |
| silicone (polydiphenyldimethylsiloxane + | |
| cyclopentadimethylsiloxane) | |
| Ascorbic acid | 1% |
| Preservatives | 1% |
| Triethanolamine | qs pH 3.5 |
| Water | qs 100% |

(*) Copolymer crosslinked with allyl methacrylate, consisting of 80% by weight of AMPS units neutralized with NH₃, and of 20% by weight of Genapol T-250 methacrylate units [units of formula (III) in which R₁═CH₃, R₄═C₁₆-C₁₈ and x = 25].

A composition is obtained which is stable and soft on application.

### Comparative Examples

In the comparative examples, Example 2 is reproduced but replacing the copolymer used according to the invention by prior art polymers: Hostacerin AMPS (AMPS homopolymer described in EP-A-815845) (sold by Hoechst-Clariant), Sepigel 305 (sold by Seppic) and Carbopol 980 (sold by Goodrich).

As shown in the table below, the use of these prior art polymers is accompanied by the observation of a high level of variation in the viscosity when 1% of acid is added (viscosity measured at approximately 25° C. using a Rheomat 180 instrument and a 3, 4 or 5 spindle depending on the viscosity range, at 200 s⁻¹) whereas in the case of the copolymer used according to the invention the viscosity remains the same.

| | | | |
|---|---|---|---|
| Example | | | |
| according to | Comparative | Comparative | Comparative |
| the invention | Example 1 | Example 2 | Example 3 |

| | no | with | no | with | no | with | no | with |
|---|---|---|---|---|---|---|---|---|
| Composition | acid | acid | acid | acid | acid | acid | acid | acid |
| Hostacerin | — | — | 2% | 2% | — | — | — | — |
| AMPS | | | | | | | | |
| Sepigel 305 | — | — | — | — | 2% | 2% | — | — |
| Carbopol 980 | — | — | — | — | — | — | 2% | 2% |
| AMPS | 2% | 2% | — | — | — | — | — | — |
| copolymer | | | | | | | | |
| according to | | | | | | | | |
| the invention | | | | | | | | |
| Mixture of | — | 1% | — | 1% | — | 1% | — | 1% |
| fruit acids | | | | | | | | |
| Water | qs | qs | qs | qs | qs | qs | qs | qs |
| | 100% | 100% | 100% | 100% | 100% | 100% | 100% | 100% |
| Viscosity | 31 | 31 | 43 | 36 | 25 | 11.5 | 85 | 66 |
| measured at | poises | poises | poises | poises | poises | poises | poises | poises |
| approximately | (3.1 | (3.1 | (4.3 | (3.6 | (2.5 | (1.15 | (8.5 | (6.6 |
| 25° C. with a | Pa · s) | Pa · s) | Pa · s) | Pa · s) | Pa · s) | Pa · s) | Pa · s) | Pa · s) |
| Rheomat 180 | | | | | | | | |
| instrument | | | | | | | | |
| using a 3, 4 | | | | | | | | |
| or 5 spindle | | | | | | | | |
| depending on | | | | | | | | |
| the viscosity | | | | | | | | |
| range, at | | | | | | | | |
| 200 s⁻¹ | | | | | | | | |

| | | | | |
|---|---|---|---|---|
| Drop in | 0% | −16% | −54% | −22% |
| viscosity | | | | |

This table shows that, contrary to the prior art polymers, the amphiphilic polymers according to the invention make it possible to prepare a composition containing an acid while retaining a viscosity which is identical to that of the composition without acid.

1. Cosmetic or dermatological composition containing a physiologically acceptable acidic aqueous medium and at least one amphiphilic polymer including at least one ethylenically unsaturated monomer containing a sulphonic group, in free form or partially or totally neutralized form and comprising at least one hydrophobic portion.

2. Composition according to claim 1, characterized in that the pH of the aqueous medium is less than or equal to 5.

3. Composition according to claim 1 or 2, characterized in that the hydrophobic portion of the amphiphilic polymer contains from 6 to 50 carbon atoms.

4. Composition according to the preceding claim, characterized in that the hydrophobic portion of the amphiphilic polymer contains from 6 to 22 carbon atoms.

5. Composition according to the preceding claim, characterized in that the hydrophobic portion of the amphiphilic polymer contains from 6 to 18 carbon atoms.

6. Composition according to the preceding claim, characterized in that the hydrophobic portion of the amphiphilic polymer contains from 12 to 18 carbon atoms.

7. Composition according to any one of the preceding claims, characterized in that the amphiphilic polymers are partially or totally neutralized with a mineral or organic base.

8. Composition according to any one of the preceding claims, characterized in that the amphiphilic polymers have a number-average molecular weight ranging from 1000 to 20,000,000 g/mol.

9. Composition according to the preceding claim, characterized in that the number-average molecular weight ranges from 20,000 to 5,000,000 g/mol.

10. Composition according to the preceding claim, characterized in that the number-average molecular weight ranges from 100,000 to 1,500,000 g/mol.

11. Composition according to any one of the preceding claims, characterized in that an aqueous solution containing 1% by weight of the said polymers has, at a temperature of 25° C., a viscosity, measured using a Brookfield viscometer with a No. 7 needle, ranging from 20,000 mpa.s to 100,000 mPa.s.

12. Composition according to any one of the preceding claims, characterized in that the amphiphilic polymers are prepared by free-radical precipitation polymerization in tert-butanol.

13. Composition according to any one of the preceding claims, characterized in that the amphiphilic polymers are crosslinked or non-crosslinked.

14. Composition according to the preceding claim, characterized in that the amphiphilic polymers are crosslinked.

15. Composition according to the preceding claim, characterized in that the crosslinking agent(s) is(are) chosen from compounds containing olefinic polyunsaturation.

16. Composition according to the preceding claim, characterized in that the crosslinking agent(s) is(are) chosen from methylenebisacrylamide, allyl methacrylate and trimethylolpropane triacrylate (TMPTA).

17. Composition according to any one of claims 14 to 16, characterized in that the degree of crosslinking preferably ranges from 0.01 mol % to 10 mol % and more particularly from 0.2 mol % to 2 mol % relative to the polymer.

18. Composition according to any one of the preceding claims, characterized in that the ethylenically unsaturated monomer containing a sulphonic group is chosen from vinylsulphonic acid, styrenesulphonic acid, (meth)acrylamido(C₁-C₂₂)alkyl-sulphonic acids, N-(C₁-C₂₂)alkyl(meth)acrylamido-(C₁-C₂₂)alkylsulphonic acids, and also partially or totally neutralized forms thereof.

19. Composition according to the preceding claim, characterized in that the ethylenically unsaturated monomer containing a sulphonic group is chosen from acrylamidomethanesulphonic acid, acrylamidoethanesulphonic acid, acrylamidopropanesulphonic acid, 2-acrylamido-2-methylpropanesulphonic acid, methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid or 2-acrylamido-2,6-dimethyl-3-heptanesulphonic acid, and also partially or totally neutralized forms thereof.

20. Composition according to either of claims 18 and 19, characterized in that the ethylenically unsaturated monomer containing a sulphonic group is 2-acrylamido-2-methylpropanesulphonic acid (AMPS), and also partially or totally neutralized forms thereof.

21. Composition according to the preceding claim, characterized in that the amphiphilic polymers are chosen from random polymers of AMPS modified by reaction with an n-mono(C₆-C₂₂)alkylamine or a di-n-(C₆-C₂₂)alkylamine.

22. Composition according to either of claims 20 and 21, characterized in that the amphiphilic polymers of AMPS also contain at least one ethylenically unsaturated monomer not comprising a fatty chain.

23. Composition according to claim 22, characterized in that the ethylenically unsaturated monomer not comprising a fatty chain is chosen from (meth)acrylic acids and β-substituted alkyl derivatives thereof, and esters thereof obtained with monoalcohols or mono- or polyalkylene glycols, or alternatively from (meth)acrylamides, vinylpyrrolidone, maleic anhydride, itaconic acid or maleic acid, or mixtures of these compounds.

24. Composition according to any one of the preceding claims, characterized in that the amphiphilic polymers are chosen from amphiphilic copolymers of AMPS and of at least one ethylenically unsaturated hydrophobic monomer comprising at least one hydrophobic portion containing from 6 to 50 carbon atoms.

25. Composition according to the preceding claim, characterized in that the hydrophobic portion contains from 6 to 22 carbon atoms.

26. Composition according to the preceding claim, characterized in that the hydrophobic portion contains from 6 to 18 carbon atoms.

27. Composition according to the preceding claim, characterized in that the hydrophobic portion contains from 12 to 18 carbon atoms.

28. Composition according to any one of claims 24 to 27, characterized in that the ethylenically unsaturated hydrophobic monomer is chosen from the acrylates or acrylamides of formula (I) below: 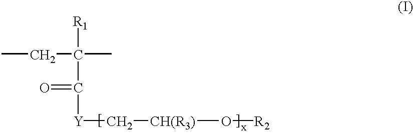
in which R₁ and R₃, which may be identical or different, denote a hydrogen atom or a linear or branched C₁-C6 alkyl radical (preferably methyl); Y denotes O or NH; R₂ denotes a hydrophobic hydrocarbon-based radical containing at least from 6 to 50 carbon atoms and more preferably from 6 to 22 carbon atoms and even more preferably from 6 to 18 carbon atoms and more particularly from 12 to 18 carbon atoms; x denotes a number of moles of alkylene oxide and ranges from 0 to 100.

29. Composition according to the preceding claim, characterized in that the hydrophobic radical R₂ is chosen from linear, branched or cyclic C₆-C₁₈ alkyl radicals; C₆-C₁₈ alkylperfluoro radicals; the cholesteryl radical or a cholesterol ester; aromatic polycyclic groups.

30. Composition according to either of claims 28 and 29, characterized in that the monomer of formula (I) also comprises at least one alkylene oxide unit (x≧1).

31. Composition according to any one of claims 28 to 30, characterized in that the monomer of formula (I) also comprises at least one polyoxyalkylenated chain.

32. Composition according to the preceding claim, characterized in that the polyoxyalkylenated chain consists of ethylene oxide units and/or of propylene oxide units.

33. Composition according to the preceding claim, characterized in that the polyoxyalkylenated chain consists solely of ethylene oxide units.

34. Composition according to any one of claims 28 to 33, characterized in that the number of moles of alkylene oxide ranges from 3 to 100.

35. Composition according to the preceding claim, characterized in that the number of moles of alkylene oxide ranges from 3 to 50.

36. Composition according to the preceding claim, characterized in that the number of moles of alkylene oxide ranges from 7 to 25.

37. Composition according to any one of claims 24 to 29, characterized in that the amphiphilic polymer of AMPS is chosen from:
crosslinked or non-crosslinked, neutralized or non-neutralized copolymers comprising from 15% to 60% by weight of AMPS units and from 40% to 85% by weight of (C₈-C₁₆)alkyl(meth)acrylamide units or of (C₈-C₁₆)alkyl (meth)acrylate units relative to the polymer;
terpolymers comprising from 10 mol % to 90 mol % of acrylamide units, from 0.1 mol % to 10 mol % of AMPS units and from 5 mol % to 80 mol % of n-(C₆-C₁₈)alkylacrylamide units relative to the polymer.

38. Composition according to any one of claims 24 to 29, characterized in that the amphiphilic polymer of AMPS is chosen from:
non-crosslinked copolymers of partially or totally neutralized AMPS and of n-dodecyl or n-hexadecyl methacrylate;
crosslinked or non-crosslinked copolymers of partially or totally neutralized AMPS and of n-dodecylmethacrylamide.

39. Composition according to claims 24 to 36, characterized in that the amphiphilic polymer of AMPS is chosen from copolymers consisting of 2-acrylamido-2-methylpropanesulphonic acid (AMPS) units of formula (II) below: 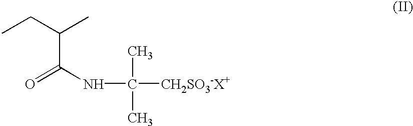
in which X⁺ is a proton, an alkali metal cation, an alkaline-earth metal cation or the ammonium ion, and of units of formula (III) below: 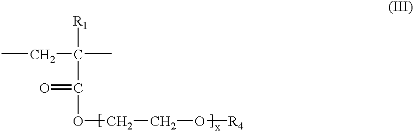
in which x denotes an integer ranging from 3 to 100, preferably from 5 to 80 and more preferably from 7 to 25; R₁ has the same meaning as that given above in formula (I) and R₄ denotes a linear or branched C₆-C₂₂ and more preferably C₁₀-C₂₂ alkyl.

40. Composition according to the preceding claim, characterized in that x=25, R₁ is methyl and R₄ is n-dodecyl, n-hexadecyl or n-octadecyl.

41. Composition according to claim 28 or 39, characterized in that the % molar proportion of units of formula (I) or of units of formula (III) in the polymers ranges from 50.1% to 99.9%.

42. Composition according to claim 28 or 39, characterized in that the % molar proportion of units of formula (I) or of units of formula (III) in the polymers ranges from 0.1% to 50%.

43. Composition according to any one of the preceding claims, characterized in that the amount of amphiphilic polymer(s) ranges from 0.01% to 30% by weight of active material, preferably from 0.1% to 10% by weight of active material, more preferably from 0.1% to 5% by weight of active material relative to the total weight of the composition.

44. Composition according to any one of the preceding claims, characterized in that the physiologically acceptable medium is composed of water or of water and at least one organic solvent selected from the group consisting of hydrophilic organic solvents, lipophilic organic solvents, amphiphilic solvents or mixtures thereof.

45. Composition according to any one of the preceding claims, characterized in that it also comprises at least one oily phase.

46. Composition according to the preceding claim, characterized in that the oily phase represents from 0.1 to 50% of the total weight of the composition.

47. Composition according to any one of the preceding claims, characterized in that it comprises at least one physiologically acceptable organic acidic active agent.

48. Composition according to the preceding claim, characterized in that the organic acidic active agent is selected from the group consisting of α-hydroxy acids, β-hydroxy acids, α-keto acids, β-keto acids, their derivatives and mixtures thereof.

49. Composition according to claim 47 or 48, characterized in that the organic acidic active agent is selected from the group consisting of ascorbic acid, kojic acid, caffeic acid, salicylic acid and its derivatives, citric acid, lactic acid, methyllactic acid, glucuronic acid, glycolic acid, pyruvic acid, 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytetradecanoic acid, 2-hydroxyhexadecanoic acid, 2-hydroxyoctadecanoic acid, 2-hydroxytetracosanoic acid, 2-hydroxyeicosanoic acid, mandelic acid, benzoic acid, phenyllactic acid, gluconic acid, galacturonic acid, aleuritic acid, ribonic acid, tartronic acid, tartaric acid, malic acid, fumaric acid, retinoic acid and its derivatives, benzene-1,4-di(3-methylidene-10-camphorsulphonic) acid, urocanic acid, 2-phenylbenzimidazole-5-sulphonic acid, α-(2-oxo-3-bornylidene)-4-toluenesulphonic acid, 2-hydroxy-4-methoxy-5-sulphonic acid, plant extracts containing acids and, more specifically, fruit extracts, acidic xanthine derivatives, β-glycyrrhetinic acid, asiatic acid, and mixtures thereof.

50. Composition according to any one of the preceding claims, characterized in that it further comprises at least one additive selected from the group consisting of hydrophilic or lipophilic, conventional gelling agents and/or thickeners; hydrophilic or lipophilic active agents; preservatives; antioxidants; perfumes; moisturizers; pigmenting agents; depigmenting agents; keratolytic agents; emulsifiers; vitamins; emollients; sequestrants; surfactants; polymers; basifying or acidifying agents; fillers; free-radical scavengers; ceramides; sunscreens; insect repellents; thinners; colorants; bactericides; and antidandruff agents.

51. Composition according to any one of the preceding claims, characterized in that it is devoid of emulsifier.

52. Composition according to any one of the preceding claims, characterized in that it constitutes a hair product used with or without rinsing, a styling product, a care and/or hygiene product, a buccodental care product or a suncare product.

53. Composition according to any one of claims 1 to 51, characterized in that it is used as a make-up product.

54. Cosmetic use of the cosmetic composition according to any one of claims 1 to 51, to treat, care for and/or make up the facial and/or body skin, mucous membranes, the scalp and/or keratin fibres.

55. Cosmetic process for treating human keratin materials, characterized in that a cosmetic composition such as defined in any one of claims 1 to 51 is applied to the human keratin materials.

56. Use of the composition according to any one of claims 1 to 51 for preparing a pommade or an ointment intended for treating the face and/or the human body, particularly for treating acne.

57. Use of an amphiphilic polymer including at least one ethylenically unsaturated monomer containing a sulphonic group, in free form or partially or totally neutralized form and comprising at least one hydrophobic portion, to obtain a stable cosmetic or dermatological composition containing at least one acidic active agent and/or having a pH less than or equal to 5.
